(19) United States
(12) Patent Application Publication
(10) Pub. No.: US 2014/0309536 A1
(43) Pub. Date: Oct. 16, 2014

(54) CATHETER WITH FLUSH VALVE AND RELATED SYSTEMS AND METHODS

(71) Applicant: LIGHTLAB IMAGING, INC., Westford, MA (US)

(72) Inventors: Nareak Douk, Lowell, MA (US); Christopher Brushett, Nashua, NH (US); Stephen M. McCartin, Chelmsford, MA (US); Hirofumi Mizoguchi, Sunnyvale, CA (US); Christopher Petersen, Carlisle, MA (US); Christopher Petroff, Groton, MA (US)

(21) Appl. No.: 14/117,781

(22) PCT Filed: Jun. 25, 2012
(86) PCT No.: PCT/US12/43969

§ 371 Date: Jul. 2, 2014

Related U.S. Application Data
(60) Provisional application No. 61/503,274, filed on Jun. 30, 2011.

(51) Int. Cl. A61B 5/00; A61M 25/09; A61M 25/00

(52) U.S. Cl. CPC A61B 5/0084; A61M 25/003; A61M 25/0015; A61B 5/0066; A61M 25/09
USPC 600/478; 600/476; 29/428

(57) ABSTRACT

In part, the invention relates to a catheter suitable for flushing a vessel. The catheter can include separated lumens and components that improve image data collection. In one embodiment, the catheter includes a catheter wall; a distal portion defining a distal lumen (62), the distal lumen having a first end terminating at the distal end of the catheter and a second end (30) terminating at an exit port in the catheter wall; a proximal portion defining proximal lumen (42), the proximal lumen having a first end terminating at the proximal end of the catheter and a second end terminating at a vent port (34) in the catheter wall; and a valve (50,54) positioned adjacent the vent port, the valve permitting fluid to exit the proximal lumen, but preventing particulate matter from the environment from entering the proximal lumen. In one embodiment, the valve comprises a piston (50) and spring (54) located in the proximal lumen (42). In another embodiment, the valve is a filter located in the proximal lumen adjacent the vent port.

### CROSS REFERENCE TO RELATED APPLICATIONS

This application claims priority to and the benefit of U.S. Provisional Patent Application No. 61/503,274 filed Jun. 30, 2011, the disclosure of which is herein incorporated by reference in its entirety.

### FIELD OF THE INVENTION

The invention relates generally to the field of catheters and more specifically to catheters suitable for collecting imaging data, fluid directing devices and other components for such catheters.

### BACKGROUND

Catheters used for optical coherence tomography (OCT) and other intraluminal imaging modalities typically include a catheter constructed for use with a guidewire and a fiberoptic element positioned in the lumen of the catheter. Light from the fiberoptic element and images of the structures illuminated by the light returned to the fiberoptic element typically pass through the walls of the catheter directly or through a transparent window in the catheter wall.

More light will leave and enter the catheter if the refractive indices of the medium inside the catheter and the fluid outside the catheter are matched. To do this, a fluid is typically introduced into the lumen of the catheter that more closely matches the fluid of the physiological site being imaged.

Certain imaging modalities such as optical coherence tomography, which are suitable for imaging tissue, are degraded when imaging through a blood field such as a blood vessel that contains blood. A need therefore exists for apparatus and methods that improve image data collection by addressing problems caused due to the presence of blood and other materials or particulates relative to a sample of interest such as a blood vessel.

### SUMMARY OF THE INVENTION

In part, the invention relates to a catheter for optical imaging and related devices, systems, components, and methods. In one embodiment, the catheter is suitable for positioning in a blood vessel near a region of interest with respect to which imaging data such as optical coherence tomography (OCT) data can be collected. The OCT data can include light reflected, scattered, or otherwise returned from a sample of interest such as a portion of a blood vessel. In one embodiment, the invention relates to stationary or moving components or subsystems of a catheter that are sized and arranged to prevent or reduce particulate matter, such as red blood cells, from degrading an image generated using an optical element in the catheter. Such components or subsystems can include one or more valves, springs, filters, membrane, slits, and other structures suitable for reducing or preventing flow of particulate matter from an environment to an optical element for collecting data within an environment such as a blood vessel.

In one aspect, the catheter includes a catheter wall; a distal portion defining a distal lumen, the distal lumen having a first end terminating at the distal end of the catheter and a second end terminating at an exit port in the catheter wall; a proximal portion defining a proximal lumen, the proximal lumen having a first end terminating at the proximal end of the catheter and a second end terminating at a vent port in the catheter wall, the proximal lumen and the distal lumen being separated and/or isolated from each other; and a valve positioned adjacent the vent port, the valve configured to permit fluid to exit the proximal lumen and prevent particulate matter from the environment from entering the proximal lumen or reducing the amount of particulate matter that reaches an imaging or optical element.

In one embodiment, the proximal lumen and the distal lumen are separated or isolated by a wall or another structure that segregates or isolates fluid in the proximal lumen and the distal lumen such that each respective fluid in each respective lumen do not mix. In another embodiment, the vent port and the exit port are adjacent one another. In yet another embodiment, the distal lumen, the distal end and the exit port are sized to accept a guidewire. In still yet another embodiment, the valve includes a piston and spring located in the proximal lumen and positioned such that when fluid in the first lumen is not under pressure the piston is biased by the spring into a first position wherein the proximal lumen is isolated from the vent port; and when fluid in the first lumen is under pressure the piston compresses the spring and moves into a second position wherein the proximal lumen is in communication with the vent port. In another embodiment, the valve is a filter located in the proximal lumen adjacent the vent port, wherein when fluid in the proximal lumen is not under pressure, fluid will move through vent port and through the filter but particulate matter is prevented from passing through the filter (or only a permissible amount passes) into the proximal lumen; and wherein when fluid in the proximal lumen is under pressure, fluid will move from the lumen through the filter and through the vent port. In yet another embodiment, the filter is a compressed spring.

In yet another aspect, the invention relates to a catheter for optical imaging. In one embodiment, the catheter includes a catheter wall having a proximal end and defining a lumen, the lumen having a first end terminating at the proximal end of the catheter and a second end terminating at a vent port in the catheter wall; and a valve positioned adjacent the vent port, the valve configured to permit fluid to exit the lumen but preventing particulate matter from the environment from entering the lumen. In another embodiment, the valve comprises a piston and spring located in the proximal lumen and positioned such that when fluid in the first lumen is not under pressure the piston is biased by the spring into a first position, wherein the proximal lumen is isolated from the vent port and when fluid in the first lumen is under pressure the piston compresses the spring and moves into a second position wherein the proximal lumen is in communication with the vent port. In still yet another embodiment, the valve is a filter located in the proximal lumen adjacent the vent port, and when fluid in the proximal lumen is not under pressure, fluid will move through vent port and through the filter. The size and arrangement of the filter is configured such that particulate matter is prevented from passing through the filter into the proximal lumen. In one embodiment, the catheter includes a proximal lumen sized and defined by a catheter wall such that when fluid in the proximal lumen is under pressure, fluid will move from the lumen through the filter and through the vent port. In still yet another embodiment, the filter is a compressed spring. In still yet another embodiment, the valve is a micro-duckbill or slit valve positioned to open and allow fluid to exit the lumen when the fluid is under sufficient pressure and to close to prevent fluid from entering the lumen from the environment when the fluid in the lumen has insufficient pressure.

In another aspect, the invention relates to a catheter for imaging such as OCT-based imaging. In one embodiment, the catheter includes a catheter wall; a distal portion defining a distal lumen, the distal lumen having a first end terminating at the distal end of the catheter and a second end terminating at an exit port in the catheter wall; a proximal portion defining a proximal lumen, the proximal lumen having a first end terminating at the proximal end of the catheter and a second end terminating at a vent port in the catheter wall, the proximal lumen and the distal lumen being separated from each other; and means for stopping flow positioned adjacent the vent port, the means for stopping flow permitting fluid to exit the proximal lumen but preventing particulate matter from the environment from entering the proximal lumen. In one embodiment, the catheter includes a guidewire channel defined by a portion of the catheter wall, the guidewire channel having a guidewire port, the guidewire port positioned such that when a guidewire is received by the catheter the vent hole is positioned under the guidewire

In yet another aspect, the invention relates to a method for preventing particulate matter from entering a lumen through a vent port. In one embodiment, the method includes the steps of placing a valve adjacent the vent port such that the valve permits fluid to pass from the lumen through the vent port, but prevents particulate matter from passing from the vent port into the lumen.

In another aspect, the invention relates to a catheter that includes a catheter wall, a distal portion defining a distal lumen, the distal lumen having a first end terminating at the distal end of the catheter and a second end terminating at an exit port in the catheter wall; a proximal portion defining a proximal lumen, the proximal lumen having a first end terminating at the proximal end of the catheter and a second end terminating at a vent port in the catheter wall, the proximal lumen and the distal lumen being separated from each other; and a filter for stopping particulate flow through the vent port into the proximal lumen, the filter positioned within the proximal lumen proximal to the vent port. In one embodiment, the filter is constructed of sintered metal.

In another aspect, the invention relates to a method of collecting optical coherence tomography data in a vessel having a vessel wall defining a vessel lumen containing particulate matter. In one embodiment, the method includes the steps of: placing an OCT probe in the vessel lumen, the OCT probe including a probe wall defining a probe lumen, the probe wall having a valve that permits fluid to pass from the probe lumen through a vent hole to the vessel lumen, but prevents particulate matter from passing from the vessel lumen through the vent hole into the probe lumen; flowing a fluid through the probe lumen and out the vent hole into the vessel lumen; and during at least a period of time when the flow of fluid is taking place through the vent hole into the vessel lumen, passing light from the OCT probe to the vessel wall while particulate matter is removed by the fluid.

In yet another aspect, the invention relates to a catheter including a catheter wall; a distal portion of the catheter wall defining a distal lumen having a first end terminating at the distal end of the catheter and a second end terminating at a first port in the catheter wall; and a proximal portion defining a proximal lumen, the proximal lumen having a first end terminating at the proximal end of the catheter and a second end terminating at a vent hole in the catheter wall, the proximal lumen and the distal lumen being separated from each other. In one embodiment, the vent hole is formed or defined by a slit in the catheter wall.

In still yet another aspect, the invention relates to a method for preventing particulate matter from entering a lumen defined by a catheter wall. In one embodiment, the method includes the steps of providing a catheter having a catheter wall, a distal portion of the catheter wall defining a distal lumen, the distal lumen having a first end terminating at the distal end of the catheter and a second end terminating at a first port in the catheter wall; and a proximal portion of the catheter wall defining a proximal lumen, the proximal lumen having a first end terminating at the proximal end of the catheter and a second end, the proximal lumen and the distal lumen being separated from each other; and placing or forming a hole such as a slit in the proximal portion of the catheter wall such that when fluid in the proximal lumen is under pressure, the slit opens, permitting fluid to pass from the proximal lumen through the slit, but when fluid in the proximal lumen is not under pressure, the slit closes preventing particulate matter from passing through the slit into the proximal lumen.

In another aspect, the invention relates to a method of collecting optical coherence tomography data in a vessel having a vessel wall defining a vessel lumen, the vessel lumen containing particulate matter. In one embodiment, the method includes the steps of placing an OCT probe in the vessel lumen, the OCT probe includes a probe wall defining a probe lumen, the probe wall having a slit that, when open, permits fluid to pass from the probe lumen through the open slit to the vessel lumen, but when closed prevents particulate matter from passing from the vessel lumen through the slit into the probe lumen; flowing a fluid through the probe lumen and out the open slit into the vessel lumen; and during a period of time when the flow of fluid is taking place through the open slit into the vessel lumen, passing light from the OCT probe to the vessel wall while particulate matter in the vessel lumen is removed by the fluid.

This Summary is provided merely to introduce certain concepts and not to identify any key or essential features of the claimed subject matter.

### BRIEF DESCRIPTION OF DRAWINGS

The objects and features of the invention can be understood more completely by referring to the drawings described below and the accompanying descriptions. In the drawings, like numerals are used to indicate like parts throughout the various views. The figures are not necessarily to scale, emphasis instead generally being placed upon illustrative principles. The figures are to be considered illustrative in all aspects and are not intended to limit the invention, the scope of which is defined only by the claims.

FIG. 1A is a top view of a catheter according to an illustrative embodiment of the invention;

FIG. 1B is a longitudinal view of section A of the catheter of FIG. 1A showing a valve in the closed position according to an illustrative embodiment of the invention;

FIG. 1C is a longitudinal view of the catheter of FIG. 1A showing the valve in the open position according to an illustrative embodiment of the invention;

FIG. 1D is a longitudinal sectional view of the catheter of FIG. 1C used with OCT optics;

FIG. 1E is photograph of the catheter of FIG. 1D;

FIG. 2A is a top view of another embodiment of a catheter according to an illustrative embodiment of the invention;

FIG. 2B is a longitudinal view of section AA of the catheter of FIG. 2A showing a valve in the closed position according to an illustrative embodiment of the invention;

FIG. 2C is a longitudinal view of the catheter of FIG. 2A showing the valve preventing the incursion of blood cells into a lumen according to an illustrative embodiment of the invention;

FIG. 3A is a longitudinal view of yet another embodiment of a catheter showing fluid being vented according to an illustrative embodiment of the invention;

FIG. 3B is a longitudinal view of the catheter of FIG. 3A with fluid not being vented according to an illustrative embodiment of the invention;

FIG. 3C is a longitudinal view of yet another embodiment of a catheter showing fluid being vented according to an illustrative embodiment of the invention;

FIGS. 4A-B are longitudinal sectional views of still yet another embodiment of a catheter showing the valve in the closed (FIG. 4A) and open (FIG. 4B) positions;

FIG. 5 is a perspective view of a catheter with a slit according to an illustrative embodiment of the invention;

FIG. 6A is a plan view of the slit of the embodiment of FIG. 5 in the closed position;

FIG. 6B is a plan view of the slit valve of the embodiment of FIG. 5 in the open position;

FIG. 6C is a side view of the slit of the embodiment of FIG. 5 in the open position in place over a guidewire;

FIG. 7 is a longitudinal section view of the slit of the embodiment of FIG. 5 in the closed position; and

FIG. 8 is a longitudinal section view of the slit of the embodiment of FIG. 5 with an included filter.

### DETAILED DESCRIPTION

The following description refers to the accompanying drawings that illustrate certain embodiments of the invention. Other embodiments are possible and modifications may be made to the embodiments without departing from the spirit and scope of the invention. Therefore, the following detailed description is not meant to limit the invention.

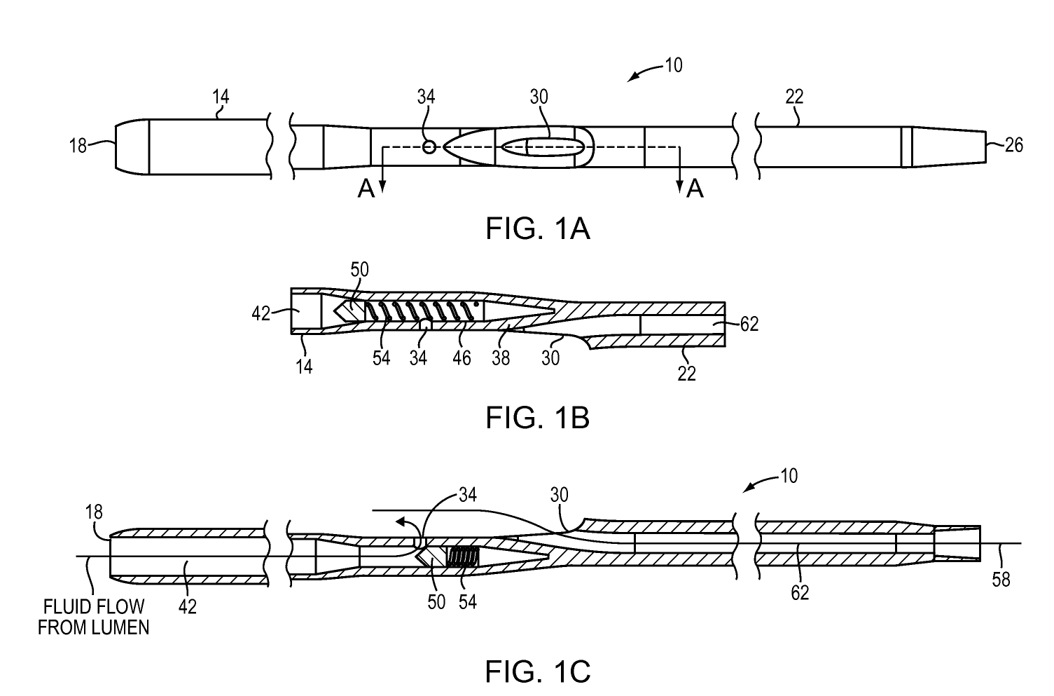

Referring to FIG. 1A, in brief overview, a catheter 10 includes a proximal portion 14 which terminates in a proximal end 18 and a distal portion 22 which terminates in a distal end 26. The distal portion 22 defines a lumen which is open at the distal end 26 and is also open at a guidewire exit port 30. The guidewire exit port 30 provides an opening to the lumen in the distal portion 22 of the catheter 10 to allow the catheter 10 to follow a guidewire that is introduced through the guidewire exit port 30 and such that the guidewire passes through the distal end 26 of the catheter 10.

In one embodiment, the catheter is made from one or more elongate or tubular sections have a plurality of varying cross-sectional thicknesses and inner diameters. The catheter can be made from any suitable material that resists shattering and can be used in an animal. The catheter can contain an imaging element.

The proximal portion 14 of the catheter 10 also includes a lumen which is open at the proximal end 18 and which includes a vent port 34 adjacent the guidewire exit port 30. The lumen and ports described herein are defined by the walls and cross-sectional geometries of the catheter in one embodiment. An optical element, not shown, such as an optical fiber with a beam director is positioned in the lumen in the proximal portion 14 of the catheter 10.

Referring also to FIG. 1B, the proximal portion 14 of the catheter 10 is separated from the distal portion 22 of the catheter 10 by a wall 38 located adjacent the guidewire exit port 30. The diameter of the lumen 42 of the proximal portion 14 is reduced near the wall 38 so as to form a cylindrical bore 46 into which is placed a piston 50 and a spring 54. Normally the spring 54 biases the piston 50 proximally, placing the piston 50 between the vent port or hole 34 and the lumen 42 of the proximal portion 14. In this position, any fluid such as blood is prevented from entering the lumen 42 and interfering with the collection of image data collection such as by obscuring one of the optical elements. Exemplary optical elements can include a lens, beam director, or rotatable optical fiber. With respect to embodiments of the invention that include a filter, structure or other element to prevent a fluid, particles, particulate matter or other matter from entering a region such as a lumen, the term “prevent,” “preventing” and similar forms or related terms includes partially preventing or regulating flow such that some of the relevant particulate matter can pass at a level or concentration that does not degrade the image data collected using the catheter.

Referring to FIG. 1C, when saline or another appropriate fluid is introduced to into the proximal portion 14 of the catheter 10 in order to purge air from the lumen 14, the pressure of the fluid in the lumen 14 increases until the piston 50 is pushed back against the spring 54, compressing the spring 54 past the vent port 34. As a result, this change in position allows the fluid in the lumen 14 to exit through the vent port 34. When the air has been purged from the catheter 10, the fluid pressure is released and the spring 54 again biases the piston 50 proximally isolating the vent port 34 from the proximal lumen 42. The continuous curved guidewire exit 30 opening and the curved lumen passage to the guidewire exit port 30 provide a smooth transition to allow the catheter to engage with and yet move smoothly along the guidewire 58.

In use, a user connects the proximal end 18 of the catheter 10 by way of a Luer-lock connector to a syringe filled with saline (not shown). When the syringe plunger is depressed, fluid passes into the lumen 42 as described above. This bolus of fluid acts as a flush that can be used to clear a blood field prior to imaging a blood vessel. When the flush is completed, the user threads the proximal end of a guidewire, which has already been positioned within, for example, a vessel to be imaged through the distal lumen 62 and out through the guidewire exit port 30. Thus positioned, the catheter 10 is then introduced into the vessel and follows the guidewire 58 into position in the vessel. Upon completion of the imaging, the catheter is pulled from the vessel and the guidewire removed.

The catheter embodiments described herein are suitable for insertion in a lumen of an animal such as an artery or other blood vessel. Imaging data such as optical coherence tomography data can be collected by an optical element disposed within a given catheter embodiment. One issue with collecting such imaging data is the presence of blood in the lumen. The diameter of red blood cells ranges from about 6 μm to about 8 μm. Accordingly, in one embodiment, the invention relates to structures such as springs, valves, membranes and other materials or structures that are sized and arranged to prevent red blood cells or other particles having a dimension ranging from about 4 μm to about 15 μm to from reaching or otherwise interfering with image data collection. In one embodiment, the data collection is performed using an imaging element such as an optical coherence tomography probe.

FIGS. 1D and 1E are a cross-sectional view of a catheter and a photograph of the catheter itself respectively used for OCT imaging. The guidewire 58 passes through the opening in the distal end 26 of the catheter, through the distal lumen 62 and out the guidewire exit port 30. The guidewire 58 passes over the vent port 34. Fluid passing from the vent port 34. Down stream from the vent port 34 is a optical assembly including a lens and optical fiber. The optical fiber and lens spins (arrow B) within the proximal lumen allowing light from the fiber to scan the blood vessel in which the catheter is positioned. Fluid passing from the proximal lumen 42 through the vent port 34 clears blood from the vessel clearing the optical field and allowing light from the fiber to pass to the vessel wall and reflected light from the vessel wall to pass back to the optical fiber unimpeded by particulate matter such as blood cells.

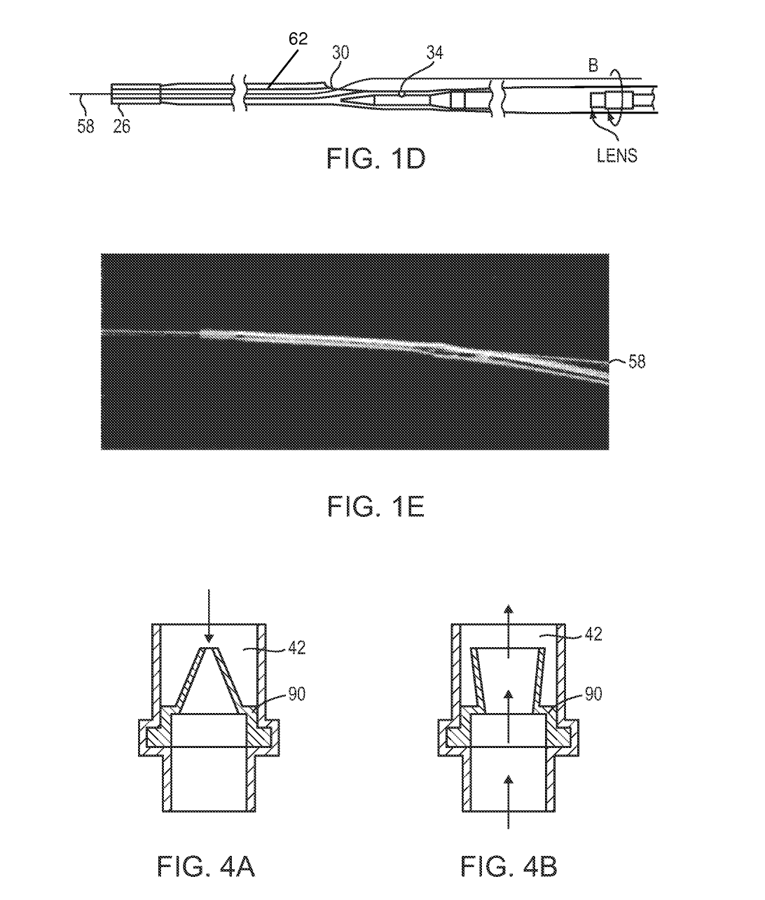

Referring now to FIG. 2A, another embodiment of a catheter 70 constructed in accordance with the invention includes a proximal portion 14 which terminates in a proximal end 18 and a distal portion 22 which terminates in a distal end 26. As shown in FIG. 2A, the distal portion 22 of this embodiment of catheter 70 includes a lumen 62, as shown in FIGS. 2A and 2C, which is open at the distal end 26 and is also open at a guidewire exit port 30. The guidewire exit port 30 provides an opening to the lumen 62 in the distal portion 22 of the catheter 70 to allow the catheter 70 to follow a guidewire 58 that exits through the guidewire exit port 30 after passing through the distal end 26 of the catheter 70.

The proximal portion 14 of the catheter 70 also includes a lumen 42 which is open at the proximal end 18 and which includes a vent port 34 adjacent the guidewire exit port 30. An optical element, such as an optical fiber with a beam director is positioned in the lumen 42 in the proximal portion 14 of the catheter 70

Referring also to FIG. 2B, the proximal portion 14 of the catheter 70 is separated or isolated from the distal portion 22 of the catheter by a wall 38 located adjacent the guidewire exit port 30. Although a wall can be used, the wall need not be the same material as the catheter and other fluid directing or block structures such as a cap, shunt, terminus, or other apparatus can be used to separate or isolate the respective lumens or portions. The diameter of the lumen 42 of the proximal catheter portion 14 is reduced near the wall 38 so as to form or define a cavity such as a cylindrical bore into which a filter 74 such as a spring or filter spring is positioned. In one embodiment, the spring has coils that are wound tightly and the filter 74 is positioned such that any particulates in the biological fluid, such as red blood cells, are prevented from entering the lumen 42 and interfering with the imaging functioning of the optical elements. The k constant, coil spacing, number of windings, material, and other features of the spring can be selected to block different species of particulate matter. In lieu of a spring, a membrane, a matrix of selectively permeable material, a valve, and other structures can be used to prevent or restrict the flow of materials that degrade image data collected using a probe disposed in the catheter.

When saline or other appropriate fluid is introduced to into the proximal portion 14 of the catheter in order to purge air from the lumen 42, the pressure of the fluid in the lumen 42 increases and the filter 74, shown as a spring embodiment, allows fluid to pass through the vent port 34. When the air has been purged from the catheter 70, the fluid pressure is released and the filter spring 74 prevents particulates in the biological fluid from entering through the vent port 34 into the proximal lumen 42 as shown in FIG. 2C.

A user of the device can connect the proximal end 18 of the catheter 70 by way of a Luer-lock connector to a syringe filled with saline (not shown). When the syringe plunger is depressed, fluid passes into the lumen 42 as described above. When the flush is completed, a user threads the proximal end of a guidewire 58, which has already been positioned within, for example, a vessel to be imaged, through the distal lumen 62 and out through the guidewire exit port 30. Thus positioned, the catheter 70 is then introduced into the vessel and follows the guidewire 58 into position in the vessel. Upon completion of the imaging, the catheter 70 is pulled from the vessel and the guidewire 58 removed.

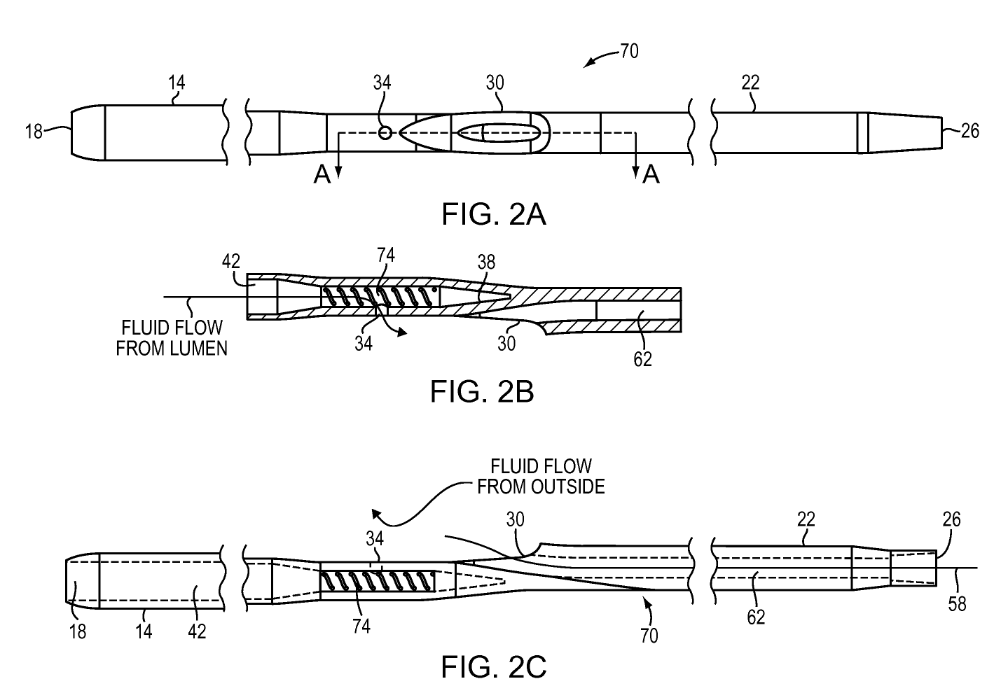

Referring now to FIG. 3A, another embodiment of a catheter 80 constructed in accordance with the invention includes a proximal portion 14 which terminates in a proximal end 18 and a distal portion 22 which terminates in a distal end 26. As shown in FIG. 3A, the distal portion 22 of this embodiment includes a lumen 62 which is open at the distal end 26 and is also open at a guidewire exit port 30. As in the previous embodiments, the guidewire exit port 30 provides an opening to the lumen 62 in the distal portion 22 of the catheter 80 to allow the catheter to follow a guidewire 58 that is introduced through the distal end 26 of the catheter and out the guidewire exit port 30.

The proximal portion 14 of the catheter 80 also includes a lumen 42 which is open at the proximal end 18 and which includes a vent port 34 adjacent the guidewire exit port 30. An optical element, such as an optical fiber with a beam director (not shown) is positioned in the lumen 42 in the proximal portion 14 of the catheter 80. In this embodiment, a flexible membrane collar 84 is secured along one edge 88 of the collar to the outside of the catheter 80 so as to cover the vent port 34. When fluid is introduced into the proximal lumen 42, FIG. 3B the pressure of the fluid causes the non-attached edge 92 of the collar 84 to move away from the outer wall of the catheter 80 and permit fluid to move from the lumen 42 out through the vent hole 34. When the purge is complete and the fluid pressure is reduced, the non-attached edge 92 of collar 84 contracts and forms a seal over the vent port 34. This seal prevents fluids from entering the proximal lumen 42.

In another embodiment, the collar 84 is attached to the catheter along both edges 88, 88′ and a slit 96 (FIG. 3C) is included in the flexible collar 84 in the region over the vent port 34 forming a slit-valve. When fluid in proximal lumen 42 is pressurized, the slit-valve 96 is forced open and fluid escapes the vent port 34. When the pressure is removed, the slit-valve 96 closes again, preventing fluid from entering the proximal lumen 42 through the vent port 34.

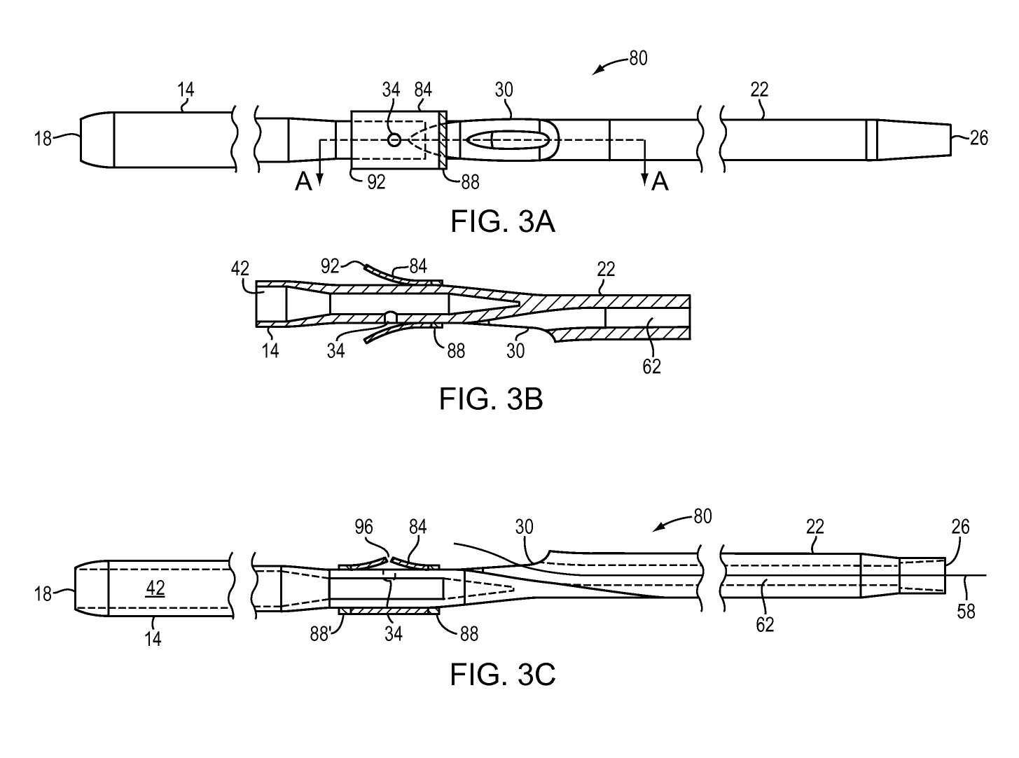

In still yet another embodiment (FIG. 4A), a micro-duckbill valve 90 is placed in the lumen 42 distal to the lens assembly (not shown). When exposed to a backflow of fluid, the duckbill closes preventing blood from entering the lumen 42. When the fluid of the lumen is under pressure the duckbill opens allowing fluid to escape (FIG. 4B).

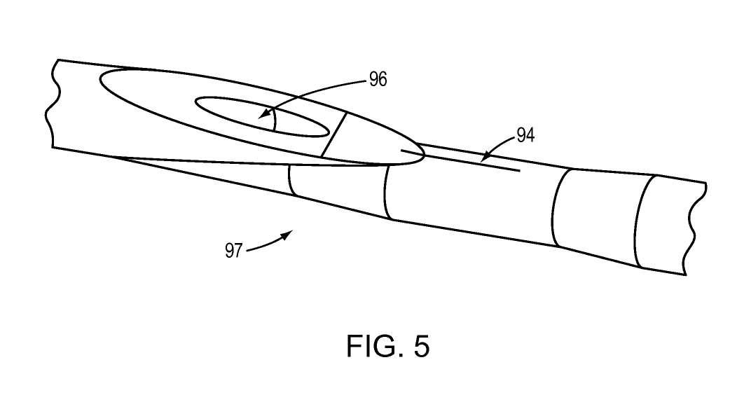

In another embodiment (FIG. 5), fluid is permitted to escape the lumen of the catheter and blood is prevented from entering the catheter 97 by means of a slit 94 cut into the outer diameter of the imaging lumen. The slit is formed as a hole in the material defining a lumen as shown. The slit 94, in one embodiment, may be formed in the wall of the catheter 97 near the opening for the guidewire 96, by a blade in either a plunge operation or a plunge and slide operation. In one embodiment, the preferred length of the slit 94, measured on the outside of the catheter, ranges from about 8 to about 1.2 mm. Blood ingress will occur with longer slits and shorter slits are difficult to purge. In one embodiment, the slit will open and close in response to pressure differences between the fluid in the lumen and the fluid in the vessel.

Once the cutting blade is removed, the slit 94 will close by itself. It remains closed in normal operation. When the slit is manufactured with a blade, due to the blade shape, the outer edge of the slit is longer than the inner edge. This does not affect the function of the slit. Other methods besides a blade may be used to create a slit including various ablation and melting devices. Since these methods remove material, an after-slitting process may be added to keep the slit completely closed in normal operation.

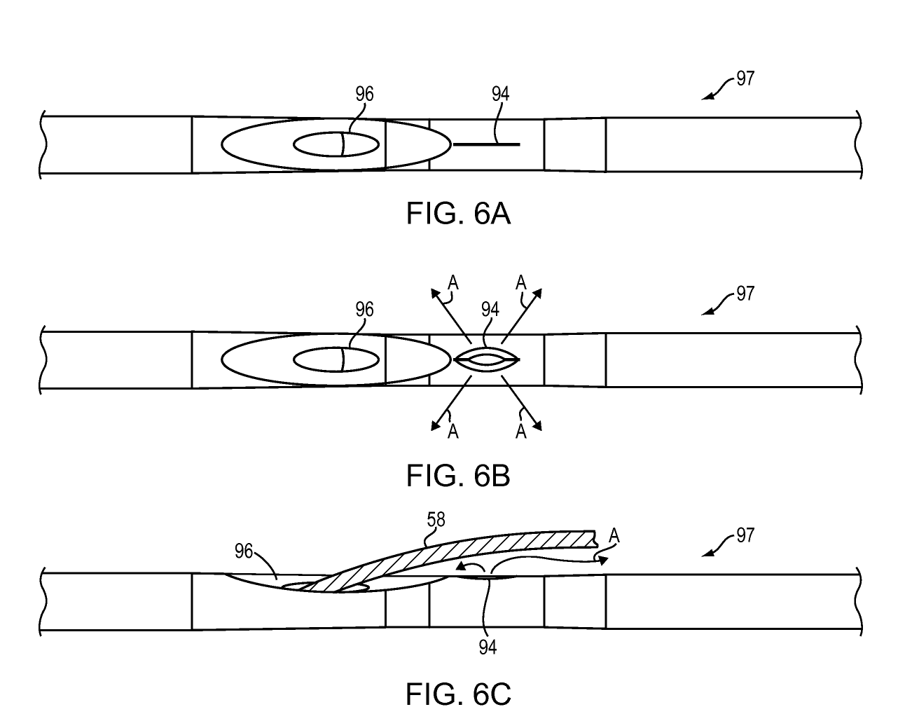

Referring also to FIGS. 6A, 6B, and 6C when fluid in a lumen of the catheter adjacent the slit is not under pressure, the slit 94 remains closed (FIG. 6A) but when the fluid is pressured and is purged from the lumen, the higher interior pressure of the purge fluid will cause the slit walls to bow open (FIG. 6B). Once the purge is complete the catheter walls will return to their as manufactured, closed shape, preventing blood from entering the catheter interior.

In various embodiments (see for example FIG. 6C) the vent port 34, when in the blood vessel, is positioned under the guidewire 58. This arrangement has various benefits. For example, this positioning permits the guidewire (not shown to clearly show the slit and the flow) to interact with the flow (arrows A) of fluid from the vent port, shown as slit 94 in this embodiment, and thereby reduce some of the pressure of the fluid impacting on the wall of the vessel, thereby lessening the chance of damage to the vessel wall.

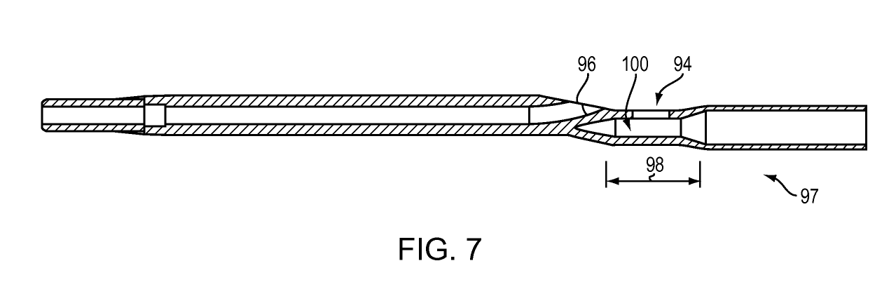

During positioning of the catheter and in tortuous vessels, torsion and bending forces on the catheter will tend to open the slit 94. As shown in FIG. 7, the wall thickness is greater in the region 98 of the slit 94. This provides more structure to the slit 94, allowing it to stay closed during handling of the catheter 97. In addition, if the catheter 97 is placed in a bent section of a vessel, the thicker wall will prevent the slit section 98 from bending and opening up the slit 94.

When the catheter 97 is purged, the purge solution displaces the air in the catheter and exits through the slit 94. The slit 94 is not directly at the end of the imaging core lumen, which creates a dead space 100 in that lumen (FIG. 7). A very small amount of air will be retained in the dead space, which is not a concern for embolism formation. The air acts as a fluid reservoir during catheter operation. When imaging components, such as a rotatable optical fiber, (not shown) is pulled back the air will momentarily expand thereby allowing the purge liquid to fill the area formerly occupied by the imaging components.

After a time, the lumen volume will be filled by liquid coming from the proximal end of catheter 97. The advance of the imaging components will either force the purge liquid out through the purge slit 94 or the purge liquid will go to the proximal end of the catheter. The air reservoir in the dead end 100 increases the effectiveness of the slit by maintaining pressure inside the catheter and reducing the chance blood will be drawn in through the slit 94 during pullback of the imaging probe or components thereof. In one embodiment, the slit simultaneously operates as a filter when no purge pressure is applied and as a purge port when pressure is applied. As a result, the use of slit eliminates the need for a separate filter element.

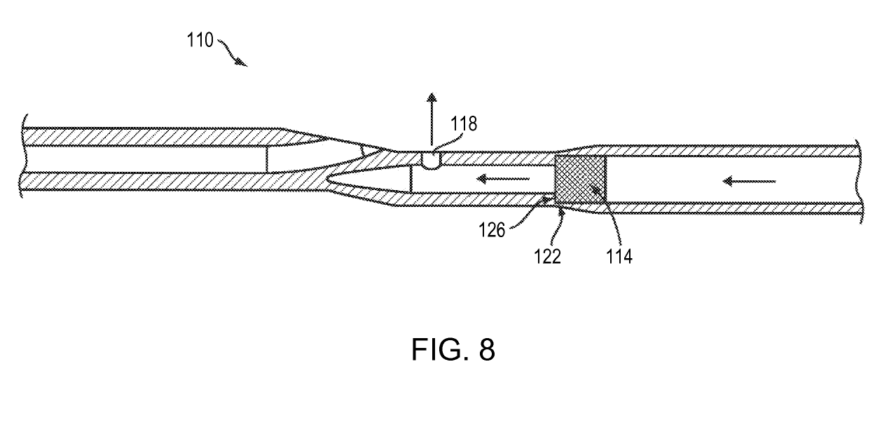

In another embodiment (FIG. 8), as an alternative to preventing all blood from returning into the catheter 110, a filter 114 may be used to allow some blood components to come back into the catheter and yet selectively stop the larger components that would adversely affect light transmission in the catheter. The larger components that are filtered typically are red blood cells. A filter 114 pore size is selected to prevent the transmission of red blood cells and larger components.

As shown in FIG. 8, in one embodiment, the filter 114 is made out of sintered metal in the shape of a cylinder. This cylinder is simply pressed into the catheter 110. Distal to the filter 114 there is an opening 118 in the catheter wall allowing communication to the blood in the vessel. The sintered metal can include holes that transmit fluid but not larger components dispersed in the fluid. A larger hole size makes it easier to purge the catheter. With the cylindrical design, the hole size may be selected to be larger than the red blood cell size and still exclude red blood cells because the long length of the filter creates a tortuous filter path that red blood cells typically will not pass through. In the preferred embodiment, the pore size of the filter 114 is about 15 μm but this may be varied over a wide range and still obtain the desired results.

When the catheter 110 is purged, the purge solution passes through the filter 114 and out the hole 118 distal to the filter. During catheter operation the pressure inside the catheter sheath will fluctuate with the motion of the imaging optical fiber. The pressure inside the sheath will drop during image acquisition when the imaging optical fiber is retracted. Liquid must enter the void created by the pulling back of the fiber either from the source of the purge fluid, the syringe on the proximal end of the catheter or from the vessel, through the filter 114. The path through the filter 114 is shorter and the pressure drop across the filter 114 may be selected to be low enough to be the preferred path for filling the void.

Under continued use of the catheter the filter 114 may become loaded with red blood cells. This will make purging more difficult. However, once in use, the catheter does not need to be purged of air and loading of the filter is not a concern. Should the clinician still desire to purge the catheter, the purge volume is much lower and purging through a loaded filter 114 is more difficult but is not a concern. In addition, (FIG. 8) the fit between the cylindrical filter 114 and the catheter sheath inner diameter 122 may be loose, allowing the sheath to expand and the purge solution to flow around the filter 114, should the filter 144 become loaded with cells. The wall thickness in the filter area 122 may be thinner to allow this to happen with lower purge pressure. Distal to the filter, the diameter may decrease 126 to prevent the filter 114 from being pushed distal during the purging operation.

In the description, the invention is discussed in the context of optical coherence tomography; however, these embodiments are not intended to be limiting and those skilled in the art will appreciate that the invention can also be used for other imaging and diagnostic modalities, instruments for interferometric sensing, or optical systems in general.

The aspects, embodiments, features, and examples of the invention are to be considered illustrative in all respects and are not intended to limit the invention, the scope of which is defined only by the claims. Other embodiments, modifications, and usages will be apparent to those skilled in the art without departing from the spirit and scope of the claimed invention.

The use of headings and sections in the application is not meant to limit the invention; each section can apply to any aspect, embodiment, or feature of the invention.

Throughout the application, where compositions are described as having, including, or comprising specific components, or where processes are described as having, including or comprising specific process steps, it is contemplated that compositions of the present teachings also consist essentially of, or consist of the recited components, and that the processes of the present teachings also consist essentially of, or consist of the recited process steps.

In the application, where an element or component is said to be included in and/or selected from a list of recited elements or components, it should be understood that the element or component can be any one of the recited elements or components and can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein.

The use of the terms “include,” “includes,” “including,” “have,” “has,” or “having” should be generally understood as open-ended and non-limiting unless specifically stated otherwise.

The use of the singular herein includes the plural (and vice versa) unless specifically stated otherwise. Moreover, the singular forms “a,” “an,” and “the” include plural forms unless the context clearly dictates otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions may be conducted simultaneously.

Where a range or list of values is provided, each intervening value between the upper and lower limits of that range or list of values is individually contemplated and is encompassed within the invention as if each value were specifically enumerated herein. In addition, smaller ranges between and including the upper and lower limits of a given range are contemplated and encompassed within the invention. The listing of exemplary values or ranges is not a disclaimer of other values or ranges between and including the upper and lower limits of a given range.

It is to be understood that the figures and descriptions of the invention have been simplified to illustrate elements that are relevant for a clear understanding of the invention, while eliminating, for purposes of clarity, other elements. Those of ordinary skill in the art will recognize, however, that these and other elements may be desirable. However, because such elements are well known in the art, and because they do not facilitate a better understanding of the invention, a discussion of such elements is not provided herein. It should be appreciated that the figures are presented for illustrative purposes and not as construction drawings. Omitted details and modifications or alternative embodiments are within the purview of persons of ordinary skill in the art.

It can be appreciated that, in certain aspects of the invention, a single component may be replaced by multiple components, and multiple components may be replaced by a single component, to provide an element or structure or to perform a given function or functions. Except where such substitution would not be operative to practice certain embodiments of the invention, such substitution is considered within the scope of the invention.

The examples presented herein are intended to illustrate potential and specific implementations of the invention. It can be appreciated that the examples are intended primarily for purposes of illustration of the invention for those skilled in the art. There may be variations to these diagrams or the operations described herein without departing from the spirit of the invention. For instance, in certain cases, method steps or operations may be performed or executed in differing order, or operations may be added, deleted or modified.

Furthermore, whereas particular embodiments of the invention have been described herein for the purpose of illustrating the invention and not for the purpose of limiting the same, it will be appreciated by those of ordinary skill in the art that numerous variations of the details, materials and arrangement of elements, steps, structures, and/or parts may be made within the principle and scope of the invention without departing from the invention as described in the claims.

What is claimed is:

1. A catheter comprising:
a catheter wall having a proximal end and a distal end;
a vent hole defined by the catheter wall;
a proximal lumen defined by the catheter wall, the proximal lumen having a first end terminating at the proximal end of the catheter and a second end terminating at the vent hole in the catheter wall; and
a valve positioned adjacent the vent hole, the valve configured to permit fluid to exit the proximal lumen and to prevent particulate matter from entering the proximal lumen.

2. The catheter of claim 1 further comprising:
a first port defined by the catheter wall; and
a distal lumen define by a distal portion of the catheter, the distal lumen comprising
a first distal portion end terminating at the distal end of the catheter and
a second distal portion end terminating at the first port defined by the catheter wall, the proximal lumen positioned such that it is isolated from the distal lumen.

3. The catheter of claim 2 wherein the proximal lumen and the distal lumen are separated by a section of the catheter wall or another fluid isolating structure.

4. The catheter of claim 2 wherein the vent hole is adjacent to the first port.

5. The catheter of claim 2, wherein the distal lumen, the distal end and the first port are sized to accept a guidewire.

6. The catheter of claim 1 wherein the valve comprises a piston and a spring located in the proximal lumen and positioned such that:
when fluid in the first lumen is not under pressure, the piston is biased by the spring into a first position, wherein the proximal lumen is isolated from the vent hole; and
when fluid in the first lumen is under pressure, the piston compresses the spring and moves into a second position, wherein the proximal lumen is in communication with the vent hole.

7. The catheter of claim 1,
wherein the valve is a filter located in the proximal lumen adjacent the vent hole,
wherein when fluid in the proximal lumen is not under pressure, fluid will move through vent hole and through the filter, but particulate matter is prevented from passing through the filter into the proximal lumen; and
wherein when fluid in the proximal lumen is under pressure, fluid will move from the lumen through the filter and through the vent hole.

8. The catheter of claim 7 wherein the filter is a spring having a plurality of coils or windings.

9. The catheter of claim 7 wherein the filter is a compressed spring.

10. The catheter of claim 7 wherein the filter comprises a sintered metal.

11. The catheter of claim 1 wherein the valve is selected from the group consisting of a filter, a coil, a membrane, a selectively permeable material, a polymer matrix, a collar, a sponge, and a plurality of holes defined by a section of catheter wall.

12. The catheter of claim 1 wherein the valve is sized and arranged to restrict the flow of red blood cells relative to an imaging probe disposed within the catheter.

13. The catheter of claim 1 wherein the valve is a micro-duckbill positioned to open and allow fluid to exit when the fluid is under sufficient pressure and to close to prevent fluid from entering when the fluid in insufficient pressure.

14. The catheter of claim 1 wherein the valve comprises means for stopping flow positioned adjacent the vent hole, the means for stopping flow permitting fluid to exit the proximal lumen and preventing particulate matter from the environment from entering the proximal lumen.

15. The catheter of claim 1 further comprising a flexible membrane attached to a section of the catheter and configured to cover the vent hole.

16. The catheter of claim 1 wherein the vent hole is a slit configured to open and seal in response to a pressure change relative to the catheter wall.

17. The catheter of claim 1 further comprising a rotatable optical fiber disposed in the proximal lumen.

18. The catheter of claim 1 further comprising a guidewire channel defined by a portion of the catheter wall, the guidewire channel having a guidewire port, the guidewire port positioned such that when a guidewire is received the vent hole is positioned under the guidewire.

19. A method for constructing a catheter comprising the steps of:
providing a catheter comprising
a catheter wall,
a distal portion of the catheter wall defining a distal lumen, the distal lumen having a first end terminating at the distal end of the catheter and a second end terminating at a first port in the catheter wall, and
a proximal portion of the catheter wall defining a proximal lumen, the proximal lumen having a first end terminating at the proximal end of the catheter,
the proximal lumen and the distal lumen being separated from each other; and
forming a hole that permits fluid to pass from the proximal lumen through the hole, but prevents particulate matter from passing from the vent hole into the proximal lumen.

20. The method of claim 19 wherein the step of forming the hole comprises placing a slit in the proximal portion of the catheter wall such that when fluid in the proximal lumen is under pressure, the slit opens, permitting fluid to pass from the proximal lumen through the slit, but when fluid in the proximal lumen is not under pressure, the slit closes preventing particulate matter from passing through the slit into the proximal lumen.

21. The method of claim 19 further comprising placing the valve adjacent the hole.
